(19) United States
(12) Patent Application Publication
(10) Pub. No.: US 2025/0099230 A1
(43) Pub. Date: Mar. 27, 2025

(54) MULTIFOCAL OPHTHALMIC LENSES

(71) Applicant: Alcon Inc., Fribourg (CH)

(72) Inventors: Wei Lu, Colleyville, TX (US); Myoung-Taek Choi, Arlington, TX (US); Zhiguang Xu, Colleyville, TX (US); Sangyeol Lee, Fort Worth, TX (US); Shinwook Lee, Arlington, TX (US); Daniel R. Carson, Forth Worth, TX (US)

(21) Appl. No.: 18/897,752

(22) Filed: Sep. 26, 2024

Related U.S. Application Data
(60) Provisional application No. 63/585,409, filed on Sep. 26, 2023.
(60) Provisional application No. 63/660,654, filed on Jun. 17, 2024.

(51) Int. Cl. A61F 2/16

(52) U.S. Cl. CPC A61F 2/1654; A61F 2/1618; A61F 2002/1696

(57) ABSTRACT

Provided in this document are examples of a multifocal ophthalmic lens. The lens includes a base lens having a base curvature corresponding to a base power; and a diffractive structure comprising a plurality of annular echelettes formed on a first surface of the base lens. The diffractive structure is configured to produce a zero-order diffraction corresponding to a distance vision focal point determined by the base power, the diffraction efficiency of the zero-order diffraction between 45% and 55%; a first-order diffraction having a diffraction efficiency between 5% and 10%; a second-order diffraction corresponding to an intermediate vision focal point, the diffraction efficiency between 15% and 20%; and a third-order diffraction corresponding to a near vision focal point, the diffraction efficiency between 15% and 25%. The diffractive structure includes a plurality of annular diffractive steps, each defined by a profile having a curved slope and a peak.

### CROSS-REFERENCE TO RELATED APPLICATIONS

The present application claims priority to U.S. Provisional Application No. 63/585,409 filed Sep. 26, 2023, entitled “MULTIFOCAL OPHTHALMIC LENSES”, and to U.S. Provisional Application No. 63/660,654 filed Jun. 17, 2024, entitled “MULTIFOCAL OPHTHALMIC LENSES”. The above-referenced applications are hereby incorporated by reference in their entirety.

### TECHNICAL FIELD

The invention set forth in the appended claims relates generally to ophthalmic devices, including, without limitation, intraocular lenses (“IOL”) for placement in the human eye.

### BACKGROUND

The human eye can suffer a variety of maladies causing mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision. While the benefits of intraocular lenses and other device implants are known, improvements continue to improve outcomes and benefit patients.

### SUMMARY

Multifocal ophthalmic lenses are disclosed, having a diffractive element comprised of a plurality of annular diffractive steps, each defined by a profile having a curved slope and a peak, and having a diffraction efficiency of at least one of the diffractive orders less than ten percent, substantially as illustrated by and described in connection with at least one of the figures, as set forth more completely in the claims.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 illustrates an intraocular lens, according to one example embodiment;

FIG. 2 depicts a side view of a surface profile of a diffractive structure on an exemplary multifocal ophthalmic lens, according to some example embodiments;

FIG. 3 depicts an energy utilization graph illustrating distribution of energy across a range of focal lengths for the exemplary surface profile of FIG. 2, according to some example embodiments;

FIG. 4 depicts visual acuity across a range of focal lengths for the exemplary surface profile of FIG. 2, according to some exemplary embodiments;

FIG. 5 depicts a graphical representation of a modulation transfer function (“MTF”) of monochromatic light across a range of focal lengths for an IOL employing the exemplary surface profile of FIG. 2, according to some exemplary embodiments; and

FIG. 6 depicts another graphical representation of a modulation transfer function (“MTF”) of monochromatic light across a range of focal lengths for an IOL employing the exemplary surface profile of FIG. 2, according to some exemplary embodiments.

### DESCRIPTION OF EXAMPLE

### Embodiments

The following description of example embodiments provides information that enables a person skilled in the art to make and use the subject matter set forth in the appended claims, but it may omit certain details already well known in the art. The following description is, therefore, to be taken as illustrative and not limiting.

The example embodiments may also be described herein with reference to spatial relationships between various elements or to the spatial orientation of various elements depicted in the attached drawings. In general, such relationships or orientations assume a frame of reference consistent with or relative to a patient. However, as should be recognized by those skilled in the art, this frame of reference is merely a descriptive expedient rather than a strict prescription.

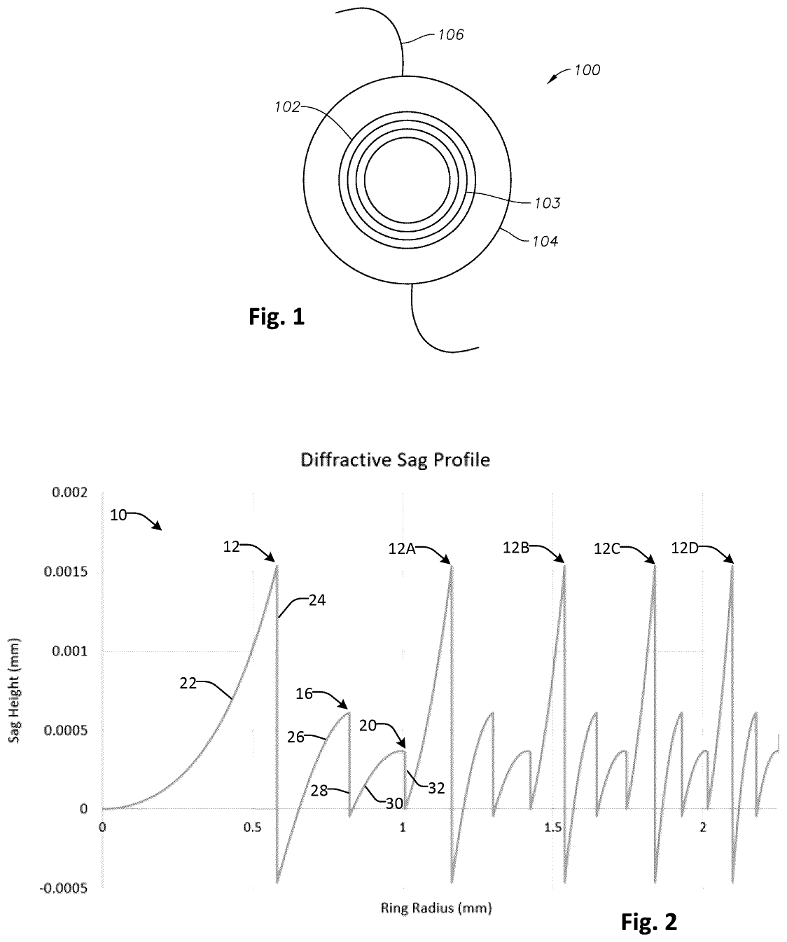

FIG. 1 illustrates an example embodiment of an ophthalmic lens having features configured to generate multiple focal points to provide a range of vision. More specifically, FIG. 1 illustrates a multifocal diffractive intraocular lens (IOL) 100. The IOL 100 may comprise an optic 104, which may include a diffractive structure 102. The IOL 100 may also comprise one or more haptics 106, which may hold the IOL in place when implanted in the eye. For example, the one or more haptics 106 may provide stable fixation of the IOL 100, and more specifically the optic 104, within the capsular bag. Although the example of FIG. 1 depicts an IOL 100 for implantation into the eye, for example in the capsular bag or the ciliary sulcus, other ophthalmic lenses incorporating a similar diffractive structure 102, including multifocal diffractive contact lenses and multifocal diffractive spectacles, are also contemplated.

The optic 104 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The optic 104 may have a diameter ϕ of between about 4.0 mm and about 8.0 mm, for example, about 6.0 mm. The optic 104 may have a variety of shapes and curvatures, which are within the scope of this disclosure. For example, the optic 104 may have an anterior surface and a posterior surface, each of which being convex, thus providing the optic 104 with a bi-convex shape. In other examples, the optic 104 may have a plano-convex shape, a convexo-concave shape, or a plano-concave shape.

The one or more haptics 106 may include hollow radially-extending struts that are coupled (e.g., glued or welded) to the peripheral portion of the optic 104 or molded along with a portion of the optic 104, and thus extend outwardly from the optic 104 to engage the perimeter wall of the capsular sac of the eye to maintain the optic 104 in a desired position in the eye. The haptics 106 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The haptics 106 may typically have radial-outward ends that define arcuate terminal portions. The terminal portions of the haptics 106 may be separated by a length L of between about 6 mm and about 22 mm, for example, about 13 mm. The haptics 106 have a particular length so that the terminal portions create a slight engagement pressure when in contact with the equatorial region of the capsular sac after being implanted. While FIG. 1 depicts one example configuration of the haptics 106, any plate haptics or other types of haptics can be used.

Referring further to FIG. 1, the IOL 100 may be a multifocal IOL (with multiple focal points, e.g., bifocal, trifocal, quadrifocal, and pentafocal) that is characterized by an optic 104 having a base curvature and a diffractive structure 102 formed on the base curvature of the optic 104. The base curvature of the optic 104 may determine a base optical power (referred to simply as a “base power”) of the IOL 100, which may correspond to a distance power for providing distance vision. Alternatively, the base power may correspond to an optical power other than the distance power, for example, an optical power corresponding to near vision or intermediate vision. The optic 104 may have an anterior surface and a posterior surface, either of both of which may have the base curvature, corresponding to the base power. In the example of FIG. 1, the diffractive structure 102 is formed on the anterior surface of the optic 104. However, in other embodiments (not shown), the diffractive structure 102 may be formed on the posterior surface or both the anterior surface and the posterior surface, or within the body of the optic 104.

The diffractive structure 102 may include a plurality of annular echelettes 103 surrounding a central region of the optic 104. For example, a first circular echelette may be centered at an optical axis of the optic 104 with a minimum radius. A second, annular echelette may be adjacent to the first circular echelette and may also be centered at the optical axis of the optic 104 with a radius larger than the minimum radius. A third annular echelette may be adjacent to the second annular echelette and may also be centered at the optical axis of the optic 104 with a radius larger than the radius of the second annular echelette. The plurality of annular echelettes 103 may include any number of echelettes, for example from about 6 to 30 echelettes. In some embodiments, the plurality of annular echelettes 103 may include from 12 to 20 echelettes, 14 to 18 echelettes, or about 15 echelettes.

Each of the plurality of annular echelettes 103 may have a sag height, or sag, that refers to a distance from the base curvature of the optic 104.

The diffractive structure 102 may divide an incoming optical energy to the anterior surface of the IOL 100 into multiple different focal points corresponding to different diffractive orders. For example, the diffractive structure 102 may diffract incident light and produce constructive interference in multiple diffractive orders associated with multiple focal points, and therefore the light energy, power, or intensity of the incident light may be divided into those multiple diffraction orders. Thus, a diffraction efficiency of each diffractive order may be less than 100%. In some exemplary embodiments, the diffractive structure 102 may divide the optical energy into at least four different focal points corresponding to different diffractive orders. The zero-order diffraction (i.e., direct transmission of light energy through the base curvature of the optic 104) may provide a distance vision determined by the base curvature of the optic 104.

In certain embodiments, one or more of the diffraction orders may not correspond to any desired focal point, and thus, the light energy that would be associated with such one or more diffraction orders may be instead allocated or distributed to one or more of the other diffractive orders, which may correspond to one or more desired focal points. For example, in some embodiments, the first-order diffraction may not correspond to any desired focal point and is instead distributed to one or more other diffractive orders. In some instances, it may be considered that the light energy corresponding to the first-order diffraction is designed to be minimal or “suppressed,” such that the energy can be distributed to one or more of the other diffraction orders. Some embodiments may additionally or alternatively provide an energy allocation between diffraction orders such that one of the diffraction orders receives a partial amount of the light energy that would be allocated to that diffraction order for providing at least some vision at a particular focal point, while still allowing for much of the energy associated with that respective diffractive order to be allocated or distributed to one or more of the other diffractive orders.

As contemplated above, in some embodiments, the diffractive structure 102 may divide incident optical energy into at least four different diffractive orders corresponding to different visual focal points. In some embodiments, the zeroth diffractive order may be used for providing distance vision, the first diffractive order may be desired to have relatively less allocated light energy as compared to the other three desired diffractive orders, or be considered partially suppressed, and the second-order diffraction and the third-order diffraction provide an intermediate vision focal point and a near vision focal point, respectively.

In some examples, the diffractive orders and/or focal points are described numerically, such as 0 order, 1^{st}, 2^{nd}, and/or 3^{rd} diffractive orders (as explained in greater detail with respect to example FIG. 2). However, descriptors are not so limited, such that the diffractive orders, and the structural and/or physical components associated therewith, could be described via any number or variety of conventions. Furthermore, some example multifocal IOLs are described herein as quadrifocal. However, the number of focal points and diffractive orders could be different (e.g., greater or smaller). For instance, some features may not be described as a diffractive order, yet contribute to the total amount of energy transmitted to the patient's eye.

In particular, some example IOLs are multifocal posterior chamber IOLs using multiple diffractive orders (i.e., 0, +1, +2, +3) with reduced or minimal light energy allocated to the +1 diffractive order. This four-order embodiment could use different consecutive diffractive orders, such as starting with an order from −4 to +2, for example. And while it may be desirable for the zero-order to be included for distance vision, that condition is not a necessary constraint for defining diffractive orders, such as diffractive orders arranged in a repeating pattern across the surface of the IOL. Lastly, the approach could be applied in principle to more than four diffractive orders; for example, a five-order diffractive lens could add powers including two intermediate powers, a near power, and a minimal energy or suppressed intermediate power.

In some embodiments, the intermediate vision focal point may be at a distance from the eye in a range of 50 cm to 80 cm, or in some more specific embodiments in a range of 60 cm to 70 cm. For example, the intermediate vision focal point may be at a distance of approximately 60 cm, which is within an optical range for performing tasks using digital screens. In some embodiments, the near vision focal point may be at a distance in a range of 30 cm to 50 cm, or in some more specific embodiments in a range of 30 cm to 40 cm. For example, the intermediate vision focal point may be at a distance of approximately 40 cm, which is an ideal distance for reading and other close-range tasks.

The distribution of the incoming optical energy (referred to as “diffraction efficiency”) to the distance vision, intermediate vision, and near vision points can be adjusted by adjusting the configurations of the annular echelettes. For example, in some embodiments, to provide desired diffraction efficiencies to the distance vision, intermediate vision, and near vision focal points, the radial spacing of the annular echelettes with respect to each other and/or the sag of each of the echelettes may be adjusted.

In some examples of a quadrifocal IOL, the first order diffraction can similarly be adjusted. As the first-order diffraction may not correspond to a desired focus in some lenses, the diffraction efficiency of the first-order diffraction may be intentionally designed to be minimal, or suppressed. This can be achieved by configuring the echelettes to limit the energy distributed to the first order diffraction, thereby allowing for more energy from incoming light to be distributed or allocated to other diffractive orders.

As disclosed herein, by adjusting the configuration of the echelettes, the total diffraction efficiency of the lens may be increased, and as a result, some additional energy from the incoming light may be allocated to the focal point associated with the first diffractive order, thereby enhancing the bridging energy between the zero-order diffraction (e.g., base focus) and second-order diffraction (e.g., intermediate focus). For example, in some embodiments, it may be desirable to allocate approximately 5-10% of the total energy intensity to the first diffractive order, in order to provide a smooth transition from distance to intermediate vision.

For example, by tailoring the amount of energy allocated to the first diffractive order, the total energy distribution across the IOL increases. Advantageously, the energy and focus in the distance, intermediate and near vision focal points remains strong. In other words, the total energy transmitted to the patient's eye is increased, while avoiding a noticeable gap between the distance and intermediate focal points. Accordingly, the patient enjoys enhanced vision while experiencing a smooth transition between distance and intermediate focal points.

In particular embodiments, the parameters are selected so that the +1 order is at least partially suppressed. In other words, the share of light energy at the +1 order is reduced relative to the division of the total light energy among the several diffraction orders intended for providing useful visual foci, such that the image at the focal point corresponding to the +1 order is no longer in clear focus. This reduced light energy amount may correspond to a light energy of less than approximately 15% of the incident light energy, or in some embodiments less than 10%. The fraction of incident light energy focused on a particular order is referred to as the “diffraction efficiency.” Thus, the diffraction efficiency at the +1 diffractive order is less than the 0, +2 and +3 diffractive orders, on the order of 10% or less.

The optical surface profile, including the diffractive structure 102, of the optic 104 may be, at least in part, defined mathematically. The optical surface profile of the multifocal lens defines the diffractive structure 102, and therefore the energy distribution and diffractive efficiency for the lens. As an example, the sag of the surface profile of the optic 104 may be defined by the following equation:

$$z = {z}_{a s p} + {z}_{d i f f}$$

where zasp and zdiff are the aspheric and the diffractive sag components, respectively.

The aspheric component (Zasp) of the surface profile of the optic 104 may be described mathematically as:

$${z}_{a s p} ( r ) = \frac{c {r}^{2}}{1 + \sqrt{1 - ( 1 + k ) {c}^{2} {r}^{2}}} , 0.0 \leq r \leq 3.0$$

where c is the curvature (i.e., inverse of radius) of a surface, k is the conic constant, and r is the radial distance from the optic center.

The diffractive component (Z_{diff}) of the surface profile of the optic 104 may be described mathematically according to the following variables, values, and equations, including those included in Tables 1-3.

TABLE 1
Constants used in Sag definition

| Name | Symbol | Value |
|---|---|---|
| Add power | D | 3.25 |
| Refractive Index of Lens | RIL | ~1.55 |
| Refractive Index of Medium | RIM | ~1.34 |
| Wavelength | λ | 0.55 |
| Trail | T | ~0.18 |

The upper border of zone n in millimeters is defined as:

$${R}_{n} = \{ \begin{matrix}0 & , n = 0 \\ \sqrt{2 n λ / D} & , n = 1 , 2 , 3 , … , 15 \\ 3. & , n = 16\end{matrix}$$

Given the distance from the optic center (r) and the zone id (n), the sag in millimeters (mm) may be calculated by the following equation:

$$sag ( r , n ) = w ( S ( n ) + H ( n ) + x ( r , n ) \cdot A ( n ) + {x ( r , n )}^{2} \cdot B ( n ) + {x ( r , n )}^{3} \cdot C ( n ) ) ,$$

where w (wave to millimeter conversion constant) and x are:

$$w = \frac{λ}{1000 ( RIL - RIM )} and x ( r , n ) = \frac{{r}^{2} - {R}_{n - 1}^{2}}{{R}_{n}^{2} - {R}_{n - 1}^{2}}$$

where A, B and C are H is phase offset; A, B and C are amplitude coefficients with numerical values between −1 and +1, S (n) is the individual phase offset for each individual echelette. Further, H, A and B define a repeating profile (e.g., three teeth or echelettes as a group, and the group repeats five times. In some examples, Stair Height(S) provides freedom to assign each peak/echelette a unique phase offset. The details of variables S, H, A, and B are listed in Tables 2 and 3.

TABLE 2
Variables used in Sag definition

| | Stair Height | |
|---|---|---|
| Zone Id (n) | (S) | Segment Id |
| 1 | 0 | 1 |
| 2 | 0 | 2 |
| 3 | 0 | 3 |
| 1 | 0 | 1 |
| 5 | 0 | 2 |
| 6 | 0 | 3 |
| 7 | 0 | 1 |
| 8 | 0 | 2 |
| 9 | 0 | 3 |
| 10 | 0 | 1 |
| 11 | 0 | 2 |
| 12 | 0 | 3 |
| 13 | 0 | 1 |
| 14 | 0 | 2 |
| 15 | 0 | 3 |
| 16 | T | 0 |

TABLE 3
Values for H, A, and B per Segment Id

| Segment Id | H | A | B |
|---|---|---|---|
| 0 | [−0.5, 0.5] | [−0.5, 0.5] | [−0.5, 0.5] |
| 1 | [−0.5, 0.5] | [0, 1] | [0, 1] |
| 2 | [−0.5, 0.5] | [0, 1] | [−1, 0] |
| 3 | [−0.5, 0.5] | [0, 1] | [−0.5, 0.5] |

FIG. 2 illustrates a side view of a diffractive sag profile 10 of an example diffractive structure 102, according to some illustrative embodiments. The exemplary diffractive sag profile of FIG. 2 may result from the sag equations and representative values discussed above. For example, the diffractive sag profile 10 is calculated from the above equation, which includes the element x (r,n)²·B (n). This may yield a curved slope in one or more diffractive echelettes, and may be present in each diffractive echelette. As a result (as provided in FIG. 2), the curvature may impact the angle of incidence at each diffractive order, which affects the amount of energy transmitted therethrough.

Curved elements used to define the amount of transmission at the first diffractive order assists in obtaining suitable bridging energy between the distance and intermediate focal points and, therefore, improved total energy utilization across all annular echelettes. For example, the above sag equation provides a sag profile with curved elements, which may repeat across the surface of the lens. Although some examples illustrate generally parabolic curves, with enhanced curvature at one or more transition points (e.g., peaks or troughs of the diffractive sag profile), in some examples any curvature may be less pronounced. Further, one or more diffractive orders may be defined by substantially linear portions. In each embodiment, the relatively limited diffraction efficiency at the first diffractive order (e.g., between the distance and intermediate focal points) aids in enhancing overall energy transmission, as well as serving as a bridge therebetween.

In FIG. 2, the horizontal axis r of the graph depicts a radial distance extending outward from a central portion of the optic 104 towards a peripheral portion or outer edge of the optic 104. The vertical axis of the graph of FIG. 2 corresponds to a sag height, or sag, for each of the echelettes. As illustrated in FIG. 2, the diffractive sag profile may include a repeating structure of 3 segments or echelettes, which may repeat over 15 zones, for a total of 15 echelettes. As previously discussed, the exemplary diffractive structure 102 may be configured to distribute light energy to four diffractive orders, resulting in the optic 104 and IOL 100 being a quadrifocal design.

As shown in FIG. 2, in some embodiments, the repeating structure of 3 echelettes may include a first echelette having a first step height 12, a second echelette having a second step height 16, and a third echelette having a third step height 20. As shown, the second step height 16 may be between a quarter and half the height of the first step height 12 of the first echelette. As further shown in FIG. 2, the third step height 20 may have a height less than the second step height 16.

Advantageously, the sag profile 10 is defined by one or more curves along a sloped or side portion. In the example of FIG. 2, a portion 22 of the sag profile 10 rises as a curve toward the first step height 12. The sag profile 10 continues as portion 24 drops at a substantially linear, substantially vertical slope, which then rises toward the second step height 16 via portion 26. Although the term linear is used to describe some portions of the sag profile and/or diffractive orders, the portion 24 (and/or portions 28, 32) may have a modest curvature, or a more pronounced curvature. Generally, the portions 22, 26 and 30 are represented as having a greater amount of curvature than portions 24, 28 and 32. As shown, the rise of portion 26 is approximately linear, becoming gradually more curved as the portion 26 approaches the next peak of the sag profile 10, at the second step height 16. Another portion 28 drops from the second step height 16, which then rises as curved portion 30 to reach the next peak at the third step height 20. In the example of FIG. 2, this pattern can be repeated, defined by curved portions rising to peak step heights, followed by substantially linear, downward portions. As shown, the distance between first step heights (e.g., between first step heights 12 and 12A, between first step heights 12A and 12B, between first step heights 12B and 12C, between first step heights 12C and 12D) decreases as the ring radius increases, providing a more condensed pattern. Thus, the curvature of the rising portions becomes sharper, in accordance with the sag equation provided herein.

As shown, the curved portions (e.g., slopes 22, 26, 30) of the sag profile are facing toward the center of the lens (e.g., toward ring radius at 0 mm). In practice, the surface of the curved portions receives incoming light corresponding to the focus of the eye's gaze. Thus, the incoming light enters the lens at the curved surface, providing a light energy distribution profile different from a substantially flat surface.

Opposite the curved surface (e.g., relative to the peaks, or step heights) is a substantially linear portion (e.g., slopes 24, 28, 32). The linear portions are facing away from the center of the lens (e.g., towards an edge of the lens). Light from objects along the periphery of the eye may enter the lens from the linear portions.

For an IOL employing the example sag profile 10, energy from an incoming light is distributed across the four diffraction orders with increased total energy utilization. The first diffraction order may receive less energy relative to the other diffraction orders, while providing a bridging energy between the zero diffraction order/distance focus and the second diffraction order/intermediate focus.

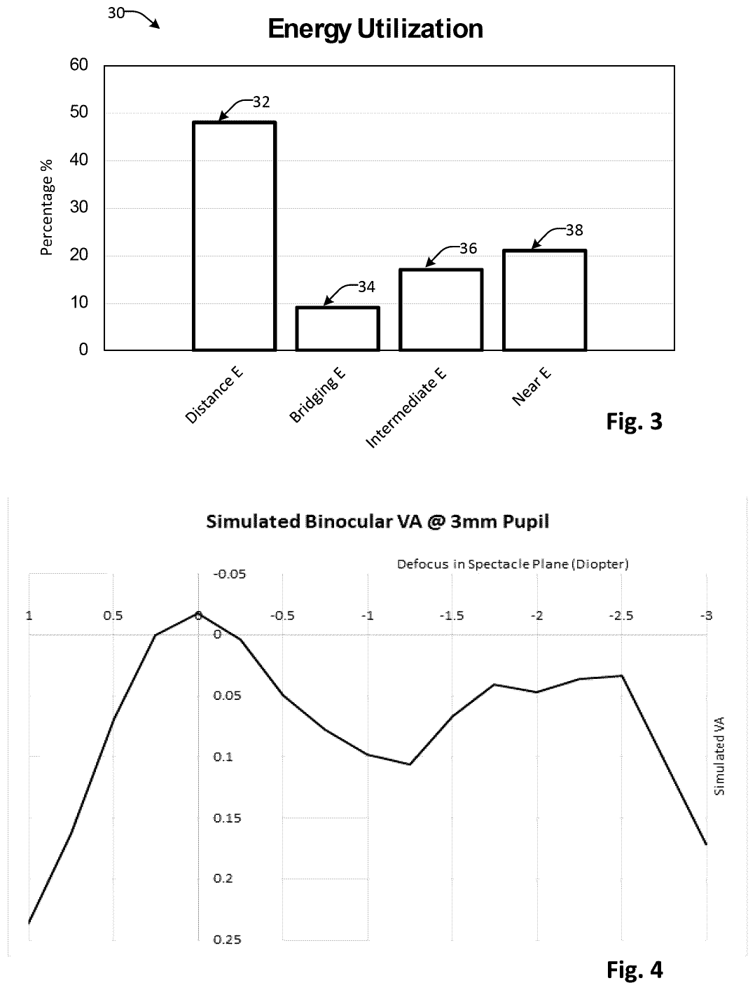

FIG. 3 depicts an energy utilization graph 30 illustrating the diffraction efficiencies calculated for the four diffractive orders corresponding to the exemplary diffractive sag surface profile 10 of FIG. 2. As illustrated in FIG. 3, four distinct energy peaks may be generated by the diffractive sag profile of FIG. 2, which correspond to the four diffractive orders (e.g., 0th diffractive order 32, 1^{st} diffractive order 34, 2^{nd} diffractive order 36, and 3^{rd} diffractive order 38). In some embodiments, approximately 40-55%, or in some embodiments 48%, of incoming light energy may be distributed to the 0^{th} diffractive order, which may correspond to distance vision. In some embodiments, approximately 15-25%, or in some embodiments 21%, of light energy may be distributed to the 3^{rd} diffractive order, which may correspond to near vision (e.g., 40 cm focal length), and approximately 10-20%, or in some embodiments 17%, of light energy may be distributed to the 2^{nd} diffractive order, which may correspond to near-intermediate vision (e.g., 60 cm focal length). Additionally, approximately 5-10%, or in some embodiments 9%, of incoming light energy may be distributed to the 1^{st} diffractive order, which may correspond to far-intermediate vision at focal lengths between distance and near-intermediate vision. While the amount of light energy distributed to the 1^{st} diffractive order may be lower than that distributed to the other diffractive orders, the light energy distributed to this 1^{st} diffractive order may function as bridging energy to provide a smooth visual transition between distance and intermediate vision. Thus, performance between the 0^{th} diffractive order and the 2^{nd} diffractive order is enhanced which may be due to the total energy utilization of the disclosed IOL being increased.

Table 4 below shows several energy ranges of the disclosed diffractive structure, in accordance with different embodiments. As provided below, several embodiments of a multifocal IOL with diffractive orders generated by the above equations were tested for energy transmission distribution. For example, an amount of energy transmitting through the diffractive structure in the below example embodiments may have a total approaching, but less than, 100%. As shown, the total energy for certain embodiments comprises some or all energy corresponding to each diffractive order for a multifocal IOL having four diffractive orders. The greatest amount of energy is allocated to the 0^{th} diffractive order (at the distance focal point), while the least amount of energy is allocated to 1^{st} diffractive order (at the bridging focal point between distance and intermediate focal points).

TABLE 4
Energy Efficiency Values (percent of total
transmitted energy per diffractive order):

| | Distance | Bridging | Intermediate | Near |
|---|---|---|---|---|
| Embodiment | Energy | Energy | Energy | Energy |
| Embodiment 1 | 48 | 9 | 17 | 21 |
| Embodiment 2 | 47 | 8 | 19 | 21 |
| Embodiment 3 | 48 | 9 | 18 | 20 |
| Embodiment 4 | 46 | 8 | 19 | 22 |

Advantageously, as a result the patient enjoys enhanced vision while experiencing a smooth transition between distance and intermediate focal points.

FIG. 4 depicts visual acuity across a range of focal lengths for the exemplary diffractive sag surface profile of FIG. 2. The visual acuity range is shown for a defocus of +1.0 D (hyperopia side) through −3.0 D (myopia side). As illustrated in the graph of FIG. 4, the diffractive sag surface profile of FIG. 2 may provide a high level of visual acuity extending from distance to near vision.

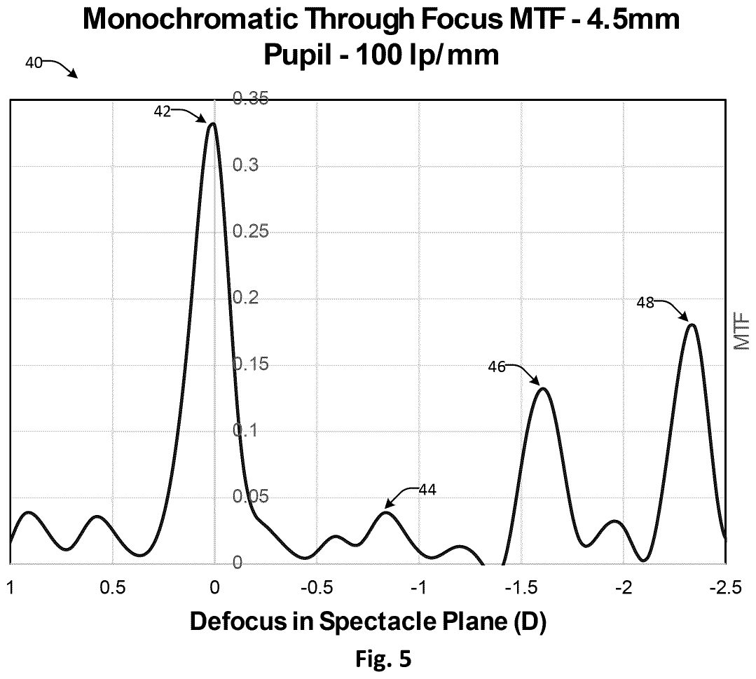

FIG. 5 depicts a graphical representation of a modulation transfer function (“MTF”) of monochromatic light across a range of focal lengths for an IOL employing the exemplary surface profile of FIG. 2, according to some exemplary embodiments. The graph 40 for a 4.5 mm pupil at 100 line pairs per millimeter (1p/mm), providing a representative measure of the resolution of the lens 100. For instance, the more line pairs per mm (1p/mm), the higher the spatial lens resolution, thus the ability to resolve finer details in an image.

As shown, each diffractive order yields a different resolution. The graph 40 illustrates four peaks representing a measure of MTF, each of which correspond to one of four distinct diffractive orders (e.g., 0th diffractive order 42, 1^{st} diffractive order 44, 2^{nd} diffractive order 46, and 3^{rd} diffractive order 48) generated by the diffractive sag profile of FIG. 2. However, in some examples, the diffraction order may not map to a number of echelettes and/or dimensions thereof. For example, several of the echelettes (e.g., two, three, four, five, six) may work together to split and/or distribute the light being transmitted to the eye into multiple diffraction orders.

In some embodiments, the MTF corresponding to the 0^{th} diffractive order is approximately 0.30-0.35, or in some embodiments 0.33. In some embodiments, the MTF corresponding to the 2^{nd} diffractive order is approximately 0.10-0.15, or in some embodiments 0.13. In some embodiments, the MTF corresponding to the 3^{rd} diffractive order is approximately 0.15-0.20, or in some embodiments 0.18. Additionally, the MTF corresponding to the 1^{st} diffractive order, which may correspond to far-intermediate vision at focal lengths between distance and near-intermediate vision, is approximately 0.01-0.06, or in some embodiments 0.04.

As explained herein, the amount of light energy distributed to the 1^{st} diffractive order, and therefore the resolution, may be lower than that distributed to the other diffractive orders, thus functioning as a (relatively muted) bridge to a smooth visual transition between distance and intermediate vision, thereby enhancing performance between the 0^{th} diffractive order and the 2^{nd} diffractive order.

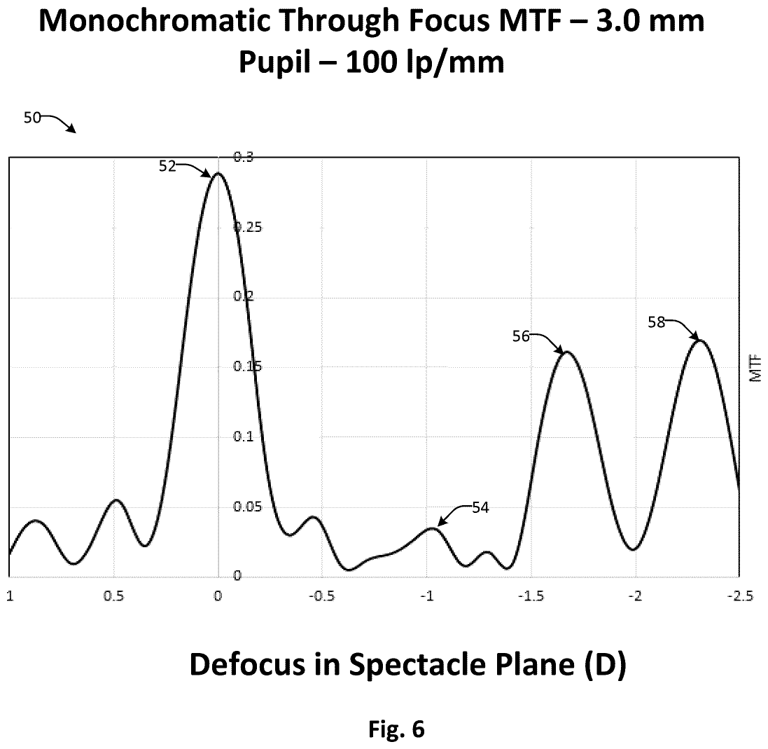

FIG. 6 depicts a graphical representation of a modulation transfer function (“MTF”) of monochromatic light across a range of focal lengths for an IOL employing the exemplary surface profile of FIG. 2, according to some exemplary embodiments. Although similar to the graph 40 of FIG. 5 (representing a MTF for a 4.5 mm pupil), FIG. 6 represents a MTF for a 3.0 mm pupil at 100 1p/mm. Thus, the graph 50 shows four peaks representing a measure of MTF, each of which correspond to one of the four distinct diffractive orders (e.g., 0th diffractive order 52, 1^{st} diffractive order 54, 2^{nd} diffractive order 56, and 3^{rd} diffractive order 58) generated by the diffractive sag profile of FIG. 2. For instance, the more line pairs per mm (1p/mm), the higher the spatial lens resolution, thus the ability to resolve finer details in an image.

In disclosed examples, a multifocal ophthalmic lens includes an optic having a base curvature corresponding to a base power; and a diffractive element, the diffractive element producing constructive interference in at least four consecutive diffractive orders, wherein the constructive interference produces a near focus, a distance focus, and an intermediate focus between the near focus and the distance focus, and wherein the diffractive element comprises a plurality of annular diffractive steps, two or more annular diffractive steps defined by a profile having a curved slope and a peak, and a diffraction efficiency of at least one of the diffractive orders is less than ten percent.

In some examples, each of the plurality of annular diffractive steps of the multifocal ophthalmic lens have a curved slope, a peak, and a linear slope.

In examples, the plurality of annular diffractive steps of the multifocal ophthalmic lens includes a repeating group of three echelettes.

In examples, a first slope of the multifocal ophthalmic lens has a curved edge approaching a peak corresponding to one or more of the diffractive steps corresponding to a diffractive sag profile generates having at least four consecutive diffractive orders.

In some examples, each of the plurality of annular diffractive steps of the multifocal ophthalmic lens has a less pronounced slope opposite the curved edge.

In some examples, the curved edge is facing a center of the multifocal ophthalmic lens.

In some examples, the linear slope is facing an outward edge of the multifocal ophthalmic lens.

In some examples, a diffraction efficiency of the first diffractive order is between five percent and nine percent.

In some examples, the diffraction efficiency of the first diffractive order is nine percent.

In some examples, the lens is an intraocular lens (IOL).

In some examples, the at least four consecutive diffractive orders are (0, +1, +2, +3).

In some examples, the diffraction efficiency of the +1 diffractive order is suppressed.

In some examples, the near focus corresponds to vision at 40 cm, and the intermediate focus corresponds to vision at 60 cm.

In some examples, the diffraction efficiency of the zero-order diffraction is between 45% and 50%; the diffraction efficiency of the first-order diffraction is between 7% and 9%; the diffraction efficiency of the second-order diffraction is at least between 15% and 20%; and the diffraction efficiency of the third-order diffraction is between 19% and 23%.

In some examples, a total energy efficiency through the diffractive element is greater than 90%.

In some disclosed examples, a multifocal ophthalmic lens includes an optic having a base curvature corresponding to a base power; and a diffractive element, the diffractive element producing constructive interference in at least four consecutive diffractive orders, wherein the constructive interference produces a near focus, a distance focus, and an intermediate focus between the near focus and the distance focus, and wherein the diffractive element has a diffractive sag profile defined by equation

$$sag ( r , n ) = w ( S ( n ) + H ( n ) + x ( r , n ) \cdot A ( n ) + {x ( r , n )}^{2} \cdot B ( n ) + {x ( r , n )}^{3} \cdot C ( n ) ) ,$$

where r is a distance from an optic center, n is a zone id, w is a wave to millimeter conversion constant, H is a phase offset, x is calculated from r, A, B and C are amplitude coefficients with numerical values between −1 and +1, S (n) is the individual phase offset for each individual echelette.

In some examples, x is calculated by equation

$$x ( r , n ) = \frac{{r}^{2} - {R}_{n - 1}^{2}}{{R}_{n}^{2} - {R}_{n - 1}^{2}}$$

where n is the index of the diffractive rings and Rn is the radius of the nth diffractive echelette.

In some examples, w is calculated by equation

$$w = \frac{λ}{1000 ( RIL - RIM )}$$

where RIL is a refractive index of the lens and RIM is refractive index of a medium that comprises the multifocal ophthalmic lens.

In some examples, the first-order diffraction has a diffraction efficiency of 8%.

In some examples, the lens is an intraocular lens (IOL).

While shown in illustrative embodiment(s), a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A multifocal ophthalmic lens, comprising:
an optic having a base curvature corresponding to a base power; and
a diffractive element, the diffractive element producing constructive interference in at least four consecutive diffractive orders, wherein the constructive interference produces a near focus, a distance focus, and an intermediate focus between the near focus and the distance focus, and
wherein the diffractive element comprises a plurality of annular diffractive steps, two or more annular diffractive steps defined by a profile having a curved slope and a peak, and
a diffraction efficiency of at least one of the diffractive orders is less than ten percent.

2. The multifocal ophthalmic lens of claim 1, wherein each of the plurality of annular diffractive steps have a curved slope, a peak, and a linear slope.

3. The multifocal ophthalmic lens of claim 2, wherein the plurality of annular diffractive steps comprises a repeating group of three echelettes.

4. The multifocal ophthalmic lens of claim 3, wherein a first slope with a curved edge approaching a peak of one or more of the diffractive steps corresponds to a diffractive sag profile having at least four consecutive diffractive orders.

5. The multifocal ophthalmic lens of claim 1, wherein each of the plurality of annular diffractive steps has a less pronounced slope opposite the curved edge.

6. The multifocal ophthalmic lens of claim 1, wherein the curved edge is facing a center of the multifocal ophthalmic lens.

7. The multifocal ophthalmic lens of claim 1, wherein the linear slope is facing an outward edge of the multifocal ophthalmic lens.

8. The multifocal ophthalmic lens of claim 1, wherein a diffraction efficiency of the first diffractive order is between five percent and nine percent.

9. The multifocal ophthalmic lens of claim 1, wherein the diffraction efficiency of the first diffractive order is nine percent.

10. The multifocal ophthalmic lens of claim 1, wherein the lens is an intraocular lens (IOL).

11. The multifocal ophthalmic lens of claim 1, wherein the at least four consecutive diffractive orders are (0, +1, +2, +3).

12. The multifocal ophthalmic lens of claim 11, wherein the diffraction efficiency of the +1 diffractive order is suppressed.

13. The multifocal ophthalmic lens of claim 1, wherein the near focus corresponds to vision at 40 cm, and the intermediate focus corresponds to vision at 60 cm.

14. The multifocal ophthalmic lens of claim 1, wherein:
the diffraction efficiency of the zero-order diffraction is between 45% and 50%; the diffraction efficiency of the first-order diffraction is between 7% and 9%;
the diffraction efficiency of the second-order diffraction is at least between 15% and 20%; and
the diffraction efficiency of the third-order diffraction is between 19% and 23%.

15. The multifocal ophthalmic lens of claim 1, wherein a total energy efficiency through the diffractive element is greater than 90%.

16. A multifocal ophthalmic lens, comprising:
an optic having a base curvature corresponding to a base power; and
a diffractive element, the diffractive element producing constructive interference in at least four consecutive diffractive orders, wherein the constructive interference produces a near focus, a distance focus, and an intermediate focus between the near focus and the distance focus, and
wherein the diffractive element has a diffractive sag profile defined by equation
$$sag ( r , n ) = w ( S ( n ) + H ( n ) + x ( r , n ) \cdot A ( n ) + {x ( r , n )}^{2} \cdot B ( n ) + {x ( r , n )}^{3} \cdot C ( n ) ) ,$$
where r is a distance from an optic center, n is a zone id, w is a wave to millimeter conversion constant, H is a phase offset, x is calculated from r, A, B and C are amplitude coefficients with numerical values between −1 and +1, S (n) is the individual phase offset for each individual echelette.

17. The multifocal ophthalmic lens of claim 16, wherein x is calculated by equation
$$x ( r , n ) = \frac{{r}^{2} - {R}_{n - 1}^{2}}{{R}_{n}^{2} - {R}_{n - 1}^{2}}$$
where n is the index of the diffractive rings and Rn is the radius of the nth diffractive echelette.

18. The multifocal ophthalmic lens of claim 16, wherein w is calculated by equation
$$w = \frac{λ}{1000 ( RIL - RIM )}$$
where RIL is a refractive index of the lens and RIM is refractive index of a medium that comprises the multifocal ophthalmic lens.

19. The multifocal ophthalmic lens of claim 16, wherein the first-order diffraction has a diffraction efficiency of 8%.

20. The multifocal ophthalmic lens of claim 16, wherein the lens is an intraocular lens (IOL).
